(19) United States
(12) Patent Application Publication
(10) Pub. No.: US 2022/0362531 A1
(43) Pub. Date: Nov. 17, 2022

(54) MICRONEEDLE PREPARATION DEVICE AND MICRONEEDLE PREPARATION METHOD

(71) Applicant: SUZHOU REVEDA MEDICAL BIOTECH CO., LTD., Suzhou, Jiangsu (CN)

(72) Inventors: Ping YAN, Shanghai (CN); Qiuyu QU, Shanghai (CN)

(21) Appl. No.: 17/771,189

(22) PCT Filed: Jan. 7, 2020
(86) PCT No.: PCT/CN2020/070725

§ 371 Date: Apr. 22, 2022

(30) Foreign Application Priority Data
Oct. 24, 2019 (CN) 201911019692.3

(51) Int. Cl. A61M 37/00; A47K 5/12

(52) U.S. Cl. CPC A61M 37/0015; A47K 5/12

(57) ABSTRACT

An apparatus and method for manufacturing a microneedle are disclosed, which aim to reduce the production costs of microneedle preparation and improve the efficiency and quality of microneedle preparation. The apparatus for manufacturing a microneedle includes a baseplate, an elastomeric female mold, a horizontal movement mechanism, a vertical movement mechanism, a dispenser and a roller mechanism. The elastomeric female mold is disposed on the baseplate and has a surface formed thereon with cavities complementary to needle bodies. The dispenser includes a reservoir for holding a solution for forming the microneedle. The roller mechanism is disposed on the dispenser and includes a roller. In practical use, the roller is driven by the vertical movement mechanism to apply a predetermined value of pressure onto the elastomeric female mold and is then driven by the horizontal movement mechanism to move horizontally on the elastomeric female mold, thereby evacuating air from the cavities and coating the solution dispensed from the reservoir on the elastomeric female mold. In this way, high filling efficiency and good filling quality are achievable.

### TECHNICAL FIELD

The present invention pertains to the field of medical devices and relates more particularly to an apparatus and method for manufacturing a microneedle.

### BACKGROUND

Most therapeutic drugs enter the human body through subcutaneous injection, an economic, rapid, direct drug delivery approach. However, patients themselves cannot use injectors easily, and the pain and fear brought about these devices further limit patient compliance. One of the solutions for this problem is to coat a drug on microneedles (including micron-sized needles) and deliver it transdermally. Transdermal delivery using microneedles allows painless drug delivery and increased patient compliance and safety. Moreover, microneedles can be used to precisely deliver a quantitative amount of a drug to a target site in a desired way. Further, microneedles can be used for skin pretreatment for enhancing skin permeability. Therefore, microneedles are promised with a good clinical application.

Microneedles can be categorized into solid and hollow, depending on their structure. In general terms, solid microneedles can increase skin permeability and pierce cell membranes, thus allowing the delivery of a drug into the blood or cells by release, permeation or otherwise. Solid microneedles may be made from a material selected from one or more of silicon, photoimageable epoxy resin, poly(methyl vinyl ether-co-maleic anhydride) (PMVE/MA), poly(lactic-co-glycolic acid) (PLGA), maltose, etc., depending on the method by which they are made. Existing techniques for manufacturing solid microneedles typically involve: preparing a polymer, a prepolymer, a monomer or a mixture of monomer as a raw material for the solid microneedles; preparing a solution with a predetermined certain viscosity from the polymer, prepolymer, monomer or mixture; and molding the microneedles by drying or crosslinking (e.g., photocrosslinking, thermal crosslinking, radiation crosslinking or chemical crosslinking). Moreover, in this approach, the molding is commonly accomplished by embossing using an elastomeric silicone female mold. Specifically, an elastomeric silicone female mold with cavities (of the same shape as the microneedles to be produced) is prepared in advance, and the solution of the polymer, prepolymer, monomer or mixture is coated on the surface of the elastomeric silicone female mold with the cavities. The mold is then placed in a vacuum environment for a period of time to allow the solution to fill up the cavities in the elastomeric silicone female mold. After that, the mold is transferred into a natural environment, and the solution is cured using a certain method (e.g., drying, crosslinking), resulting in the formation of the microneedles.

However, in the above manufacturing techniques, the mold has to be placed in a vacuum environment in order to evacuate air from the cavities in the elastomeric silicone female mold so that the solution of the polymer, prepolymer, monomer or mixture can sufficiently fill the cavities. Moreover, when the solution is highly viscous, it may not be able to fill the cavities in the elastomeric silicone female mold due to insufficient evacuation of air therefrom. This may possibly lead to failure in forming the microneedles. Further, in order to provide the vacuum environment, the manufacturing system has to adopt a complicated design and compromise on continuous operability and hence on productivity.

### SUMMARY OF THE INVENTION

It is an object of the present invention to provide an apparatus and method for manufacturing a microneedle, which allow increased structural simplicity of the apparatus, lower manufacturing cost, fewer defects in manufactured microneedles, improved microneedle manufacturing efficiency and higher quality of manufactured microneedles.

To this end, the present invention provides an apparatus for manufacturing a microneedle, the microneedle including a substrate and a number of needle bodies formed on the substrate, the apparatus for manufacturing a microneedle including:

a baseplate;

an elastomeric female mold disposed on the baseplate, the elastomeric female mold having a surface where cavities complementary to the needle bodies of the microneedle are formed;

a dispenser including a reservoir, the reservoir configured to contain a solution for forming the microneedle;

a roller mechanism arranged on the dispenser, the roller mechanism including a roller rotatable about an axis of rotation;

a horizontal movement mechanism configured to drive the dispenser to move horizontally; and a vertical movement mechanism configured to drive the dispenser to move vertically,

wherein: the reservoir includes a closed configuration and an open configuration; in the closed configuration, the reservoir encloses a space and prevents dispensing therefrom of the solution for forming the microneedle, and in the open configuration, the space of the reservoir is opened, allowing dispensing therefrom of the solution for forming the microneedle;

in the open configuration of the reservoir, the roller is configured to apply a predetermined value of pressure onto the elastomeric female mold under the drive of the dispenser that is being driven by the vertical movement mechanism and to then move horizontally on the elastomeric female mold and thus evacuate air from the cavities and coat the solution dispensed from the opened space on the elastomeric female mold under the drive of the dispenser that is instead being driven by the horizontal movement mechanism.

Optionally, the roller mechanism may further include a roller drive assembly connected to the roller, the roller drive assembly configured to drive the roller to rotate about the axis of rotation, thus creating rolling friction between the roller and the elastomeric female mold.

Optionally, the apparatus may further include a controller communicatively connected to, and configured to control movement of, each of the vertical movement mechanism, the horizontal movement mechanism, the roller mechanism and the dispenser.

Optionally, the dispenser may be mounted on the vertical movement mechanism which may be in turn mounted on the horizontal movement mechanism,

wherein the horizontal movement mechanism is configured to drive the vertical movement mechanism and the dispenser to move in a first horizontal direction which is perpendicular to both the axis of rotation of the roller and a vertical direction.

Optionally, the horizontal movement mechanism may be further configured to drive the vertical movement mechanism and the dispenser to move in a second horizontal direction which is parallel to the axis of rotation of the roller.

Optionally, the horizontal movement mechanism may include a horizontal movement member, a frame structure and a first driver, the frame structure disposed on the horizontal movement member, the horizontal movement member configured to be driven by the first driver to move horizontally together with the frame structure, the frame structure configured to cause horizontal movement of the dispenser.

Optionally, the apparatus may further include a controller communicatively connected to the first driver, the controller configured to control a mode of operation of the first driver.

Optionally, the vertical movement mechanism may include a vertical movement member, a second driver and a support, the support disposed on the vertical movement member, the vertical movement member configured to be driven by the second driver to move vertically together with the support, the support configured to cause vertical movement of the dispenser.

Optionally, the apparatus may further include a controller communicatively connected to the second driver, the controller configured to control a mode of operation of the second driver.

Optionally, the dispenser may be rotatably connected to the vertical movement mechanism.

Optionally, the horizontal and vertical movement mechanisms may be gantry/carriage assemblies with X- and Z-directional movability, or parts of gantry/carriage assemblies with movability along three axes.

Optionally, the dispenser may include a top casing member, a fixed baffle and a plurality of movable baffles, the fixed baffle arranged adjacently to the movable baffle, the fixed baffle fixed to the top casing member, at least one of the movable baffles movably disposed on the top casing member, the roller rotatably arranged on the fixed baffle so as to be opposed to the top casing member, the top casing member, the fixed baffle, the roller and the movable baffles together forming the reservoir,

wherein in the closed configuration of the reservoir, each of the movable baffles is in sealed contact with the roller; and

wherein in the open configuration of the reservoir, at least one of the movable baffle(s) movably disposed on the top casing member is spaced away from the roller.

Optionally, the apparatus may include two movable baffles, which may be disposed on opposite sides of the axis of rotation of the roller.

Optionally, the dispenser may further include a horizontal adjustment assembly connected to at least one of the two movable baffles, the horizontal adjustment assembly configured to adjust a horizontal distance between the two movable baffles to cause a change in the configuration of the reservoir.

Optionally, the horizontal adjustment assembly may include a biased element and a horizontal driver,

the horizontal driver configured to overcome a force exerted by the biased element to cause relative movement of the two movable baffles, the biased element configured to store potential energy during the relative movement of the two movable baffles caused by the horizontal driver, the biased element further configured to release the stored potential energy when the horizontal driver stops acting on the two movable baffles, thus cause reverse relative movement of the two movable baffles.

Optionally, the apparatus may further include a controller communicatively connected to the horizontal driver, the controller configured to control a mode of operation of the horizontal driver.

Optionally, the biased element may be a compression spring and the horizontal driver may include a direct-acting solenoid, the direct-acting solenoid including an output shaft connected to the movable baffle, the compression spring disposed over the output shaft of the direct-acting solenoid in such a manner that its one end abuts against the direct-acting solenoid and the other end is connected to the movable baffle.

Alternatively, the dispenser may further include a side casing member disposed outside the movable baffle, with the biased element being implemented as a compression spring and with the horizontal driver including a direct-acting solenoid, the direct-acting solenoid including an output shaft connected to one side of the movable baffle, the movable baffle connected at the opposite side to one end of the compression spring, the other end of the compression spring connected to the side casing member.

Optionally, the horizontal adjustment assembly may include a linkage mechanism and a linkage driver for driving the linkage mechanism to move, the linkage mechanism configured to cause at least one of the movable baffles to horizontally move away or toward the roller.

Optionally, the dispenser may further include a height adjustment assembly connected to the movable baffles, the height adjustment assembly configured to adjust a vertical gap between at least one of the movable baffles and the elastomeric female mold, which defines a thickness of the substrate of the microneedle.

Optionally, the movable baffle may include a first baffle element and a second baffle element, the second baffle element located closer to the elastomeric female mold, the second baffle element vertically movably disposed on the first baffle element, wherein the height adjustment assembly is arranged on the first baffle element and configured to drive the second baffle element to move vertically, thus adjusting the vertical gap between the at least one movable baffle and the elastomeric female mold.

Optionally, the height adjustment assembly may include a gear/rack drive mechanism for driving the second baffle element to move vertically relative to the first baffle element.

Optionally, the first baffle element may have a cavity in which the gear/rack drive mechanism is accommodated, and an upper portion of the second baffle element may be received in the cavity of the first baffle element and connected to a rack in the gear/rack drive mechanism.

Alternatively, the first baffle element may have a cavity and the gear/rack drive mechanism may be arranged on an external side wall of the first baffle element spaced away from the reservoir, with an upper portion of the second baffle element being inserted in the first baffle element, or with the second baffle element being movably arranged adjacent to an internal side way of the first baffle element close to the reservoir.

Optionally, the height adjustment assembly may further include a height driver for electrically driving the gear/rack drive mechanism to move.

Alternatively, the height adjustment assembly may further include a rotary adjustment knob disposed outside the first baffle element, the rotary adjustment knob connected to a gear in the gear/rack drive mechanism and configured to be manually manipulated to drive the gear/rack drive mechanism to move.

Optionally, the apparatus may further include a controller communicatively connected to the height driver, the controller configured to control a mode of operation of the height driver.

Optionally, the dispenser may further include a height adjustment assembly connected to the movable baffle, the height adjustment assembly including a height driver, slide rails and a tumbler, the movable baffle consisting of a plurality of slats, an upper portion of the movable baffle wrapped around the tumbler, the remaining lower portion of the movable baffle disposed slidably in the slide rails, the tumbler transmissively connected to the height driver so as to be driven thereby to drive the movable baffle to move in a direction defined by the slide rails.

Optionally, the apparatus may further include a pressure sensor for sensing pressure on the elastomeric female mold and producing pressure information for use in the detennination of whether the pressure on the elastomeric female mold reaches a predetermined pressure value.

Optionally, the apparatus may further include a controller communicatively connected to the pressure sensor, the controller configured to determine, based on the pressure information, whether the pressure on the elastomeric female mold reaches the predetermined pressure value, and if so, control the vertical movement mechanism to stop moving downward, thereby keeping the current pressure on the elastomeric female mold.

To the above end, the present invention also provides a method for manufacturing a microneedle, which is based upon an apparatus for manufacturing the microneedle. The apparatus includes a baseplate, an elastomeric female mold, a dispenser, a roller mechanism, a horizontal movement mechanism and a vertical movement mechanism, the elastomeric female mold disposed on the baseplate, the elastomeric female mold having a surface where cavities complementary to needle bodies of the microneedle are formed, the dispenser including a reservoir for containing a solution for forming the microneedle, the reservoir including a closed configuration and an open configuration, the roller mechanism arranged on the dispenser and including a roller rotatable about an axis of rotation. The method includes:

bringing the reservoir into the open configuration and thereby dispensing the solution for forming the microneedle;

driving, by the vertical movement mechanism, the roller to vertically move downward and apply a predetermined value of pressure to the elastomeric female mold; and

driving, by the horizontal movement mechanism, the roller to horizontally move on the elastomeric female mold, thereby evacuating air from the cavities of the elastomeric female mold and coating the dispensed solution on the elastomeric female mold.

Optionally, the method may further include:

monitoring the pressure on the elastomeric female mold and producing pressure information, by the pressure sensor, during the application of the pressure to the elastomeric female mold by the roller; and

controlling the pressure on the elastomeric female mold by causing the vertical movement mechanism, based on the pressure information, to continue or stop moving downward.

Optionally, the dispenser may include a top casing member, a fixed baffle and two movable baffles, at least one of the movable baffles movably disposed on the top casing member, the two movable baffles disposed on opposite sides of the roller,

wherein the step of bringing the reservoir into the open configuration and thereby dispensing the solution for forming the microneedle includes:

driving the movable baffle movably disposed on the top casing member to move away from the roller, thus bringing the reservoir into the open configuration and allowing the solution to flow out of the reservoir onto the surface of the elastomeric female mold via a resulting gap between the movable baffle and the roller.

Optionally, the dispenser may further include a height adjustment assembly, wherein the step of bringing the reservoir into the open configuration and thereby dispensing the solution for forming the microneedle further includes:

adjusting a vertical gap between the at least one movable baffle and the elastomeric female mold by the height adjustment assembly, thus defining a thickness of a substrate of the microneedle.

Optionally, the method may further include, subsequent to the horizontal movement of the roller on the elastomeric female mold driven by the horizontal movement mechanism,

removing the other solution for forming the microneedle than that that has formed the needle bodies and, before or after it is dried, joining a substrate to the needle bodies, thereby forming the microneedle.

Optionally, the method may further include, prior to the step of bringing the reservoir into the open configuration and thereby dispensing the solution for forming the microneedle,

bringing the reservoir into the closed configuration and driving the dispenser by the horizontal movement mechanism and/or the vertical movement mechanism to move until the roller comes into contact with the surface of the elastomeric female mold.

The above apparatus and method have at least one of the following advantages:

First, during the manufacturing of the microneedle, the roller presses and deforms the elastomeric female mold to adequately evacuate air from the cavities of the elastomeric female mold. In particular, the roller squeezes elastomeric female mold while coating the solution dispensed from the opened space onto the elastomeric female mold. In this way, high filling efficiency and good filling quality are achievable. Moreover, the use of vacuuming equipment is dispensed with, greatly simplifying the apparatus and reducing the manufacturing cost. Further, continuous production is allowed, resulting in improved production efficiency.

Second, the reservoir of the dispenser can be opened and closed. Containing the solution for forming the microneedle in this reservoir enables ease of use and results in an additional increase in filling efficiency of the solution.

Third, during the manufacturing of the microneedle, the vertical gap set by the height adjustment assembly between the movable baffle and the elastomeric female mold allows precise control of a coating thickness of the solution on the female mold surface and thus of the substrate thickness of the microneedle, improving processing accuracy of the apparatus and method.

Fourth, during the manufacturing of the microneedle, the roller disposed between the two movable baffles can divide the solution into two streams, one in front of the roller and the other behind the roller along the direction in which the roller is advancing. The solution dispensed in front of the roller can be driven by the roller into the cavities, and the solution dispensed behind the roller can be filled back into the cavities. In this way, even high filling efficiency and even better filling quality, of the solution, can be achieved.

### BRIEF DESCRIPTION OF THE DRAWINGS

Those of ordinary skill in the art would appreciate that the following drawings are presented merely to enable a better understanding of the present invention rather than to limit the scope thereof in any sense. In the drawings:

FIG. 1 is a structural schematic of an apparatus for manufacturing a microneedle according to a preferred embodiment of the present invention;

FIG. 2 explains how a microneedle is manufactured in accordance with a preferred embodiment of the present invention;

FIG. 3 is a structural schematic of a dispenser according to a preferred embodiment of the present invention;

FIG. 4 is a partial cross-sectional view of the dispenser in an open configuration according to a preferred embodiment of the present invention; and

FIG. 5 is a partial cross-sectional view of the dispenser in the apparatus in a closed configuration according to a preferred embodiment of the present invention.

In these figures,

10—apparatus for manufacturing a microneedle;

11—vertical movement mechanism; 111—support;

12—horizontal movement mechanism; 121—frame structure; 122—base;

13—roller mechanism; 131—roller; 132—roller drive assembly; 14—elastomeric female mold;

141—cavity; 15—baseplate;

16—dispenser; 161—reservoir; 162—fixed baffle; 163—movable baffle; 1631—first baffle element; 1632—second baffle element; 164—gear/rack drive mechanism; 165—rotary adjustment knob; 166—return spring; 167—direct-acting solenoid; 168—liquid supply pipeline; 169—liquid level sensor;

17—controller; 18—solution; 19—pressure sensor.

### DETAILED DESCRIPTION

The present invention will be described in greater detail below with reference to the accompanying schematic drawings, which present preferred embodiments of the invention. It would be appreciated that those skilled in the art can make changes to the invention disclosed herein while still obtaining the beneficial results thereof. Therefore, the following description shall be construed as being intended to be widely known by those skilled in the art rather than as limiting the invention.

For the sake of clarity, not all features of an actual implementation are described in this specification. In the following, description and details of well-known functions and structures are omitted to avoid unnecessarily obscuring the invention. It should be appreciated that in the development of any such actual implementation, numerous implementation-specific decisions must be made to achieve specific goals of the developers, such as compliance with system-related and business-related constrains, which may vary from one implementation to another. Moreover, it should be appreciated that such a development effort might be complex and time consuming, but would nevertheless be a routine undertaking for those of ordinary skill in the art.

The present invention will be described in greater detail below by way of examples with reference to the accompanying drawings. Advantages and features of the present invention will become more apparent from the following description and from the appended claims. Note that the figures are provided in a very simplified form not necessarily drawn to exact scale for the only purpose of facilitating easy and clear description of the disclosed embodiments.

In embodiments of the present invention, there is provided a method for manufacturing a microneedle, in particular a solid microneedle, more in particular an integrally-formed solid microneedle. The microneedle includes a substrate and a number of microneedles formed on the substrate. In particular, the microneedle is manufactured using an apparatus according to the present invention, which in particular includes a baseplate, an elastomeric female mold, a dispenser, a roller mechanism, a horizontal movement mechanism and a vertical movement mechanism. The elastomeric female mold is arranged on the baseplate, and cavities complementary to the microneedles are formed in a surface of the elastomeric female mold. The dispenser includes a reservoir for holding a solution for forming the microneedle. The reservoir includes a closed configuration and an open configuration. The roller mechanism is disposed on the dispenser and includes a roller rotatable about an axis of rotation. The horizontal movement mechanism is configured to drive the dispenser to move horizontally, and the vertical movement mechanism is configured to drive the dispenser to move vertically.

The method for manufacturing a microneedle is implemented by the apparatus and includes:

bringing the reservoir into the open configuration, thereby dispensing the solution for manufacturing the microneedle;

the vertical movement mechanism driving the roller (in particular, driving the dispenser and hence the roller) to move vertically downward and apply a predetermined value of pressure onto the elastomeric female mold; and

the horizontal movement mechanism driving the roller (in particular, driving the dispenser and hence the roller) to move horizontally on the elastomeric female mold to evacuate air from the cavities in the elastomeric female mold and coat the dispensed solution on the elastomeric female mold.

Specifically, in practical applications, the vertical movement mechanism first drives the roller to move downward to apply a predetermined value of pressure onto the elastomeric female mold, causing elastic deformation of the elastomeric female mold and thus adequate evacuation of air from the cavities thereof. Immediately following that, the horizontal movement mechanism drives the roller to move horizontally on the elastomeric female mold. At this point, the roller is preferred to roll relative to the elastomeric female mold (i.e., there is rolling friction between the elastomeric female mold and the roller). In this manner, the dispensed solution is driven by the rolling action into the cavities in a timely manner immediately following the evacuation of air therefrom. This approach consisting of simultaneous squeezing of the elastomeric female mold by the roller and timely driving of the solution into the cavities as a result of the roller's horizontal movement on the elastomeric female mold has the advantages of high filling efficiency and good filling quality of the solution, eliminated use of vacuuming equipment, a greatly simplified structure of the manufacturing apparatus, reduced manufacturing cost, continuous operability and high productivity.

Further description is set forth below with reference to the accompanying drawings.

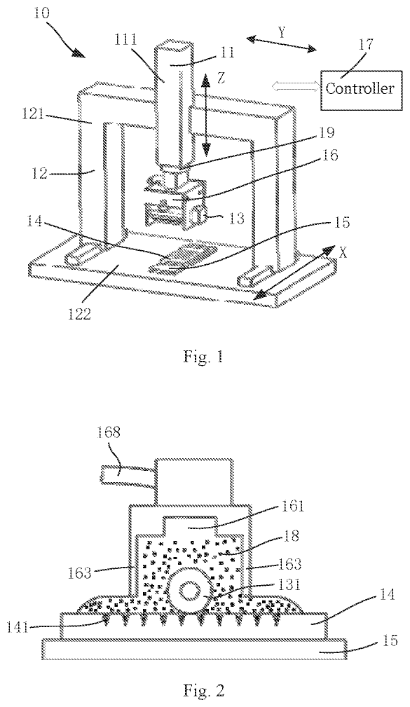

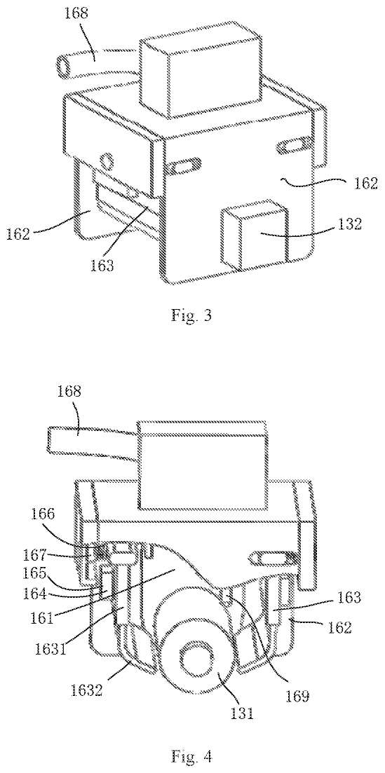

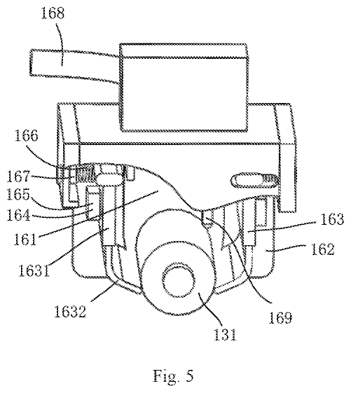

FIG. 1 is a structural schematic of an apparatus for manufacturing a microneedle according to a preferred embodiment of the present invention. FIG. 2 explains how a microneedle is manufactured in accordance with a preferred embodiment of the present invention. FIG. 3 is a structural schematic of a dispenser according to a preferred embodiment of the present invention. FIG. 4 is a partial cross-sectional view of the dispenser in an open configuration according to a preferred embodiment of the present invention. FIG. 5 is a partial cross-sectional view of the dispenser in the apparatus for manufacturing a microneedle in a closed configuration according to a preferred embodiment of the present invention. As shown in FIGS. 1 to 5, the apparatus 10 is for manufacturing a microneedle, in particular, a solid microneedle, includes a substrate and a number of microneedles formed on the substrate. Specifically, the apparatus 10 includes a vertical movement mechanism 11, a horizontal movement mechanism 12, a roller mechanism 13, an elastomeric female mold 14, a baseplate 15 and the dispenser 16.

Preferably, the apparatus 10 for manufacturing a microneedle further includes a controller 17 for controlling movement of the other components. Specifically, the controller 17 is communicatively coupled to each of the vertical movement mechanism 11, the horizontal movement mechanism 12, the roller mechanism 13 and the dispenser 16, in particular to electrical elements in these components, and is thus able to control moving parts in them to operate in an automated manner to more efficiently manufacture the microneedle. The present invention is not limited to any particular controller 17, and the controller 17 may be implemented as a hardware component for performing logical operations, such as a single-chip microcomputer, a microprocessor, or a programmable logic controller (PLC) or a field programmable logic gate array (FPGA), or as a software program, functional module, function, object library or dynamic-link library capable of performing the above functions on the basis of hardware, or as a combination of both. Based on the teachings disclosed herein, those skilled in the art will know how to enable the controller 17 to communicate with the other components. Although it has been described above to preferably use the controller 17, those skilled in the art may achieve the same technical effects otherwise, e.g., by manual or mechanical control.

The elastomeric female mold 14 is an elastic material which imparts good ability to recover from deformation to the elastomeric female mold 14 (i.e., good elasticity). Preferably, the material of the elastomeric female mold 14 is silicone. As shown in FIG. 2, a number of cavities 141 are formed in a surface of the elastomeric female mold 14. The cavities 141 are the same as the microneedles of the solid microneedle being manufactured in terms of shape, size, quantity and arrangement. A non-limiting example of the shape of the cavities 141 is regular square pyramidal.

The elastomeric female mold 14 is disposed on the baseplate 15 in such a manner that the baseplate 15 rigidly supports the elastomeric female mold 14. The baseplate 15 is preferably formed of a rigid material. However, the present invention is not limited to any particular rigid material, as long as the baseplate 15 does not deform or deform only to a negligible extent during operation of the roller mechanism 13. In other embodiments, the baseplate 15 may be formed of a flexible material with desirable rigidity. For example, it may be a sufficiently rigid silicone plate, which can support the elastomeric female mold 14 to some extent while not affecting the deformability of the rolled elastomeric female mold 14. Additionally, the elastomeric female mold 14 is detachably secured to the baseplate 15. For example, the elastomeric female mold 14 may be clamped at opposite ends by a fixture which is spaced apart from the cavities 141 in the elastomeric female mold 14 by margins for avoiding the fixture from interfering with the dispenser 16 in operation.

As shown in FIGS. 3, 4 and 5, the roller mechanism 13 includes a roller 131 rotatable about an axis of rotation. As a key component of the manufacturing apparatus, the roller 131 is configured both to squeeze and elastically deform the elastomeric female mold 14 to evacuate air from the cavities 141 and to drive the dispensed solution 18 into the cavities 141 in a timely manner so that the dispensed solution 18 adequately fills the cavities 141. Preferably, the roller mechanism 13 further includes a roller drive assembly 132 coupled to the roller 131. The roller drive assembly 132 is configured to drive the roller 131 to rotate about the axis of rotation to avoid dry friction between the roller 131 and the elastomeric female mold 14. Preferably, the roller drive assembly 132 is also communicatively coupled to the controller 17.

As shown in FIG. 1, the roller mechanism 13 is mounted on the dispenser 16, the dispenser 16 is in turn on the vertical movement mechanism 11, and the vertical movement mechanism 11 is in turn on the horizontal movement mechanism 12. In alternative embodiments, the vertical movement mechanism 11 may not be mounted on the horizontal movement mechanism 12. Preferably, the controller 17 is configured to control the horizontal movement mechanism 12 so as to cause it to drive both the vertical movement mechanism 11 and the dispenser 16 to move in a first horizontal direction. The first horizontal direction is defined as a direction perpendicular to both the axis of rotation of the roller 131 and a vertical direction. In this document, the first horizontal direction is sometimes interchangeably referred to as an “X direction”. The controller 17 is also configured to control the vertical movement mechanism 11 so as to cause it to drive the dispenser 16 to move in the vertical direction. In this document, the vertical direction is sometimes interchangeably referred to as a “Z direction”. More preferably, the controller 17 is further configured to control the horizontal movement mechanism 12 so as to cause it to drive both the vertical movement mechanism 11 and the dispenser 16 to move in a second horizontal direction that is parallel to the axis of rotation of the roller 131. In this document, the second horizontal direction is sometimes interchangeably referred to as a “Y direction”.

The present invention is not limited to any particular material of the roller 131. Preferably, the roller 131 is made of stainless steel, which imparts good corrosion resistance and polishing properties to the roller 131. More preferably, the stainless steel roller 131 has a smoother surface resulting from an electrolytic polishing process, which can reduce damage brought to the elastomeric female mold.

The present invention is not limited to any roller drive assembly 132, and one skilled in the art can choose a proper one according to the actual assembly requirements. For example, the roller drive assembly 132 may include a stepper motor and a decelerator. In this case, the stepper motor may be coupled to the roller 131 via the decelerator. The roller drive assembly 132 is also communicatively coupled to the controller 17 so that the controller 17 can control operation of the roller 131 by activating or deactivating the roller drive assembly 132.

Referring to FIGS. 4 and 5, in conjunction with FIG. 2, the dispenser 16 includes a reservoir 161 for holding the solution 18 for forming the microneedle. The solution 18 has a predetermined dynamic viscosity preferably of 5,000-20,000 cps. The present invention is not limited to any particular solution 18. Preferably, the solution 18 is an aqueous solution of a polymer, a prepolymer, a monomer or a mixture of monomers. The reservoir 161 includes a closed configuration and an open configuration. For example, in the closed configuration, the reservoir 161 may enclose a space in which the solution 18 is contained. In the open configuration, the space of the reservoir 161 may be opened to allow the solution 18 to be dispensed therefrom. Preferably, the reservoir 161 may transition between the closed and open configurations under the control of the controller 17, allowing ease of use and high efficiency of filling the solution. Preferably, the roller 131 of the roller mechanism 13 is disposed in the reservoir 161. In this way, with the aid of the roller 131, not only the reservoir 161 can be easily closed, but also splitting of the solution is enabled.

With continued reference to FIGS. 3 to 5, the dispenser 16 further includes a top casing member (not labeled), a fixed baffle 162 and a movable baffle 163. The fixed baffle 162 is disposed in neighborhood of the movable baffle 163 and secured to the top casing member. At least one movable baffle 163 may be movably arranged on the top casing member. In addition, the roller 131 is rotatably disposed on the fixed baffle 162 so as to be opposed to the top casing member. Further, the top casing member, the fixed baffle 162, the roller 131 and the movable baffle 163 together define the reservoir 161. In the closed configuration of the reservoir 161, the movable baffle 163 remains in contact with the roller 131, and the space of the reservoir 161 is enclosed. In the open configuration of the reservoir 161, the at least one movable baffle 163 that is movably disposed on the top casing member is separated from the roller 131, opening the space of the reservoir 161. In the illustrated embodiment, the roller 131 constitute a base for the reservoir 161, and two movable baffles 163 are disposed on opposite sides of the axis of rotation of the roller 131.

Further, the dispenser 16 further includes a horizontal adjustment assembly coupled to the at least one movable baffle 163. The horizontal adjustment assembly is configured to adjust a horizontal distance between the two movable baffles 163, resulting in a change in the configuration of the reservoir. In some embodiments, one of the movable baffles 163 can move, and the other movable baffle 163 is kept stationary. In such embodiments, the horizontal adjustment assembly is coupled to one of the movable baffles. In some embodiments, both of the movable baffles 163 can move. In such embodiments, the horizontal adjustment assembly is coupled to both of the movable baffles 163. In the illustrated embodiment, both of the movable baffles 163 can move. The reservoir 161 can be opened as a result of the horizontal adjustment assembly driving the two movable baffles 163 to move away from each other and can be closed as a result of the horizontal adjustment assembly driving the two movable baffles 163 to move toward each other. In the closed configuration, the two movable baffles 163 are both in sealed contact with the roller 131. Further moving the two movable baffles 163 away from each other will form gaps between the respective movable baffles 163 and the roller 131, one in front of the roller and the other behind the roller along the direction in which the roller is rolling and advancing. The solution 18 can flow out from the gaps in front of and behind the roller. In the illustrated embodiment, the roller 131 is at least partially disposed within the reservoir 161, with its opposing ends rotatably resting on the fixed baffle 162.

Additionally, the horizontal adjustment assembly may include a biased element and a horizontal driver. The horizontal driver may be configured to overcome forces exerted by the biased element to cause the two movable baffles 163 to move away from each other, thus bringing the reservoir 161 into the open configuration. The biased element may be configured to store potential energy while the horizontal driver is driving the two movable baffles 163 to move away from each other and to release the stored potential energy after the horizontal driver stops acting on the two movable baffles 163, which causes the two movable baffles 163 to move toward each other until they come into sealed contact with the roller 131 and thus close the reservoir 161. Alternatively, the horizontal driver may be configured to overcome forces exerted by the biased elements to cause the two movable baffles 163 to move toward each other and come into sealed contact with the roller 131, thus bringing the reservoir 161 into the closed configuration, with the biased element being configured to store potential energy while the horizontal driver is driving the two movable baffles 163 to move toward each other and to release the stored potential energy after the horizontal driver stops acting on the two movable baffles 163, which causes the two movable baffles 163 to move away from each other and thus open the reservoir 161. Preferably, the horizontal driver is also communicatively coupled to the controller 17 so as to be able to change its mode of operation under the control of the controller 17. Examples of the mode of operation may include activation, deactivation and variation of revolution speed and direction.

Optionally, the biased element may be a compression spring 166, and the horizontal driver may include a direct-acting solenoid 167. In one example, an output shaft of the direct-acting solenoid 167 is connected to the movable baffle 163, and the compression spring 166 is disposed over the output shaft of the direct-acting solenoid 167 with its one end abutting against the direct-acting solenoid 167 and the other end connected to the movable baffle 163. In exemplary embodiments with two movable baffles 163, each movable baffle 163 may be connected to a respective compression spring 166 disposed over a respective direct-acting solenoid 167 (e.g., over an output shaft of the direct-acting solenoid 167). One end of the compression spring 166 may abut against the direct-acting solenoid 167, while its other end may be connected to the movable baffle 163. The direct-acting solenoids 167 may be fixed to the top casing member. When the direct-acting solenoids 167 are energized under the control of the controller 17, their output shafts may push the movable baffles 163 away from the roller 131, thus causing them to move away from each other. The present invention is not limited to any particular structure or model of the direct-acting solenoid 167. In an alternative embodiment, the dispenser 16 may further include a side casing member (not labeled) disposed outside the movable baffle 163.In this case, the output shaft of the direct-acting solenoid 167 may be connected to one side of the movable baffle 163, and the compression spring 166 may be connected to the opposite side of the movable baffle 163. Additionally, the other end of the compression spring 166 may be connected to the side casing member.

More specifically, referring to FIGS. 2, 4 and 5, the direct-acting solenoid 167 may exert a force on the movable baffle 163 to push the movable baffle 163 horizontally away from the roller 131. As a result, a horizontal gap may be created between the bottom of the movable baffle 163 (i.e., the second baffle element 1632 described below) and the roller 131, allowing the solution 18 to flow out of the reservoir 161. In this process, the compression spring 166 may store potential energy. Additionally, when the direct-acting solenoid 167 is deenergized to stop acting on the movable baffle 163, the compression spring 166 may release the potential energy, which causes the two movable baffles 163 to move toward each other until the second baffle element 1632 comes into contact with the roller 131 and encloses a space together with the fixed baffle 162, in which the solution 18 is contained and sealed, preventing leakage of the solution 18 when the dispenser 16 is out of operation.

In an alternative embodiment, the combination of the compression spring 166 and the direct-acting solenoid 167 is replaced with a linkage mechanism and a linkage driver for driving the linkage mechanism to move. The linkage mechanism is configured to horizontally push or pull the movable baffle 163 away from or toward the roller 131. The linkage mechanism may be, for example, a slider-crank mechanism. The linkage driver may be a mechanical power source such as a motor or a pneumatic cylinder. These implementations are also within the scope of the present invention. The present invention is not limited to any particular implementation of the horizontal adjustment assembly.

The dispenser 16 further includes a height adjustment assembly coupled to the movable baffle 163 and configured to adjust a vertical gap (or vertical distance) between the at least one movable baffle 163 and the elastomeric female mold 14, which determines a thickness of the substrate of the solid microneedle. In other words, a coating thickness of the solution 18 on the elastomeric female mold 14 depends on the vertical gap set by the height adjustment assembly between the movable baffle 163 and the elastomeric female mold 14, and the final substrate thickness of the resulting solid microneedle is determined by both the coating thickness and the properties of the solution (e.g., the presence of water or not, the degree of shrinkage depending on the polymerization of the monomer(s) contained therein). Preferably, the height adjustment assembly is also communicatively coupled to the controller 17.

In the illustrated embodiment, the height adjustment assembly is configured to change a vertically length of the movable baffle 163 by vertically shortening or elongating the movable baffle 163. As shown in FIGS. 4 and 5, the movable baffle 163 that can be shortened or elongated may include a first baffle element 1631 and a second baffle element 1632 which is disposed on the first baffle element 1631 so as to be lower and closer to the elastomeric female mold 14 than, and movable vertically relative to the first baffle element 1631, to shorten or elongate the movable baffle 163. Moreover, the height adjustment assembly is arranged on the first baffle element 1631 and is configured to drive the second baffle element 1632 to move vertically to adjust the vertical gap between the movable baffle 163 and the elastomeric female mold 14.

In one embodiment, only one movable baffle 163 is changeable in vertical length, and the coating thickness of the solution can only be set on one side of the roller 131. In this case, the apparatus 10 has to operate in a one-way manner in which the dispenser 16 must return to a start point after each coating cycle before it can start the next coating operation. In an alternative embodiment, two movable baffles 163 are provided and both can be changed in vertical length. Specifically, the height adjustment assembly is coupled to both the movable baffles 163 so as to be able to adjust vertical gaps between each movable baffle and the elastomeric female mold. With this arrangement, the coating thickness of the solution can be set on both sides of the roller 131, enabling the apparatus 10 to work in a two-way manner without needing to return to a start point after each coating cycle. This allows for higher operating efficiency compared to the one-way arrangement. Further, in order to attain a uniform thickness of the substrate, the vertical gaps between the movable baffles 163 and the elastomeric female mold 14 are desired to be equal. Preferably, the second baffle elements 1632 are curved in shape in order to reduce resistance to flow of the solution and better guide the flow, which can result in greater ease of use.

Optionally, the height adjustment assembly may include a gear/rack drive mechanism 164 (see FIGS. 4 and 5) for driving the second baffle element 1632 to vertically move relative to the first baffle element 1631. Preferably, the first baffle element 1631 has a cavity in which the gear/rack drive mechanism 164 is accommodated, and at least part of the second baffle element 1632 (e.g., an upper portion thereof) extends in the cavity of the first baffle element 1631 and is coupled to a rack in the gear/rack drive mechanism 64. Moreover, in order to prevent leakage of the solution, an upper portion of the second baffle element 1632 is sealed against a lower portion of the first baffle element 1631, e.g., by a seal strip. Optionally, the height adjustment assembly may further include a rotary adjustment knob 165, which is disposed outside the first baffle element 1631 and is coupled to a gear in the gear/rack drive mechanism 14. The rotary adjustment knob 165 may be manually operated to cause rotation of the gear in the gear/rack drive mechanism 164, which will in turn cause translational movement of the rack. In other embodiments, the rotary adjustment knob 165 may be replaced by a height driver (e.g., a motor). In the case, the height driver may electrically drive the gear/rack drive mechanism 1644, and the controller 17 may be communicatively coupled to the height driver so as to be able to control movement of the gear/rack drive mechanism 16 via the height driver. In alternative embodiments, in order to simplify the manufacturing process, the gear/rack drive mechanism 164 may be disposed on an external side wall of the first baffle element 1631 spaced away from the reservoir 161, with an upper portion of the second baffle element 1632 being movably inserted in the cavity of the first baffle element 1631, or with the second baffle element 1632 being arranged in neighborhood of an internal side wall of the first baffle element 1631 adjacent to the reservoir 161.

As described above, when one of the movable baffles 163 cannot be varied in length, the apparatus 10 has to operate in a one-way manner in which the dispenser 16 can coat the solution along only one direction. Specifically, after each coating cycle, the controller 17 controls the horizontal adjustment assembly to close the reservoir 161 and then controls the vertical movement mechanism 11 to drive the dispenser 16 to move upward. After that, it controls the horizontal movement mechanism 12 to drive the dispenser 16 to horizontally move back to a start point. Finally, it controls the vertical movement mechanism 11 to cause the dispenser 16 to move down into contact with the roller 131 and then squeeze the elastomeric female mold 14. Subsequently, the controller 17 controls the horizontal movement mechanism 12 to drive the dispenser 16 to begin the next coating cycle from the start point.

In an alternative embodiment, the height adjustment assembly may be implemented to work like a rolling shutter. In this case, it may include a height driver, slide rails and a tumbler. Correspondingly, the movable baffle 163 may consist of a set of hinged slats. Additionally, a top portion of the movable baffle 163 may be wrapped around the tumbler, and the remaining lower portion may be disposed slidably in the slide rails. The tumbler may be transmissively coupled to the height driver so as to be able to be driven thereby to drive the movable baffle 163 to move in a direction defined by the slide rails.

Preferably, the dispenser 16 is able to spin in order to increase flexibility and adaptability of the manufacturing apparatus and facilitate the positioning of the elastomeric female mold 14. For example, the dispenser 16 may be rotatably coupled to the vertical movement mechanism 11. Further, a spin drive means (e.g., a motor) for driving the dispenser 16 to spin may be mounted on the vertical movement mechanism 11. In embodiments of the apparatus 10 with one movable baffle 163 incapable of being varied in length, two-way coating is made possible with the spinnable dispenser 16.

Referring back to FIG. 1, the horizontal movement mechanism 12 includes a frame structure 121, horizontal movement members and a first driver (not shown). The frame structure 121 is disposed on the horizontal movement members, which can be driven by the first driver to horizontally move while carrying the frame structure 121. The frame structure 121 is configured to drive the dispenser 16, e.g., through the vertical movement mechanism 11, to move horizontally. The present invention is not limited to any particular horizontal movement member. For example, it may be a slider/track assembly, a gear/rack assembly, or a ball/screw assembly. The first driver may be a motor, or a hydraulic or pneumatic drive, for example. Preferably, the horizontal movement mechanism 12 further includes a base 122 on which the horizontal movement member resides. Further, the baseplate 14 may also be disposed on the base 122. Alternatively, the base 122 may be directly used as the baseplate 15.

The vertical movement mechanism 11 includes a vertical movement member, a support 111 and a second driver (not shown). The support 111 is arranged on the vertical movement member so that the vertical movement member can move with the support 111 vertically when driven by the second driver. The support 111 is coupled to the dispenser 16 so as to be able to cause vertical movement of the dispenser 16. Additionally, the vertical movement member is disposed on the horizontal movement mechanism 12, e.g., on the frame structure 121 of the horizontal movement mechanism 12. The present invention is not limited to any particular vertical movement member. For example, it may be a slider/track assembly, a gear/rack assembly, or a ball/screw assembly. The second driver may be a motor, or a hydraulic or pneumatic drive, for example. Preferably, both the first and second drivers are communicatively coupled to the controller 17 so that the controller 17 can control modes of operation of the first and second drivers (e.g., activation, deactivation, variation of revolution speed or direction, or the like). More specifically, the activation, deactivation, variation of revolution speed and direction and the like of the first and second drivers may be done in response to receipt of commands from the controller 17.

In alternative embodiments, the functions of both the horizontal movement mechanism 12 and the vertical movement mechanism 11 may be provided by a single mechanism, without departing from the scope of the present invention. For example, the horizontal movement mechanism 12 and the vertical movement mechanism 11 may be replaced by gantry/carriage assemblies with X- and Z-directional movability. Alternatively, the horizontal movement mechanism 12 and the vertical movement mechanism 11 may be replaced by gantry/carriage assemblies with movability along three axes. The added horizontal degree of freedom can increase the scope of application of the manufacturing apparatus.

The apparatus 10 further includes a pressure sensor 19 for sensing pressure on the elastomeric female mold 14 and producing pressure information. Preferably, the controller 17 is communicatively coupled to the pressure sensor 19. In practical use, the vertical movement mechanism 11 drives the dispenser 16 to move down until the roller 131 comes into contact with the elastomeric female mold 14 and applies a pressure thereto. The pressure sensor 19 senses the pressure and feeds a value of the pressure as pressure information to the controller 17. The controller 17 then determines, based on the pressure information, whether the current pressure on the elastomeric female mold 14 reaches a predetermined value. If so, the controller 17 controls the vertical movement mechanism 11 to stop further moving downward to keep the pressure. If not, the controller 17 controls the vertical movement mechanism 11 to continue moving downward until the predetermined pressure value is achieved. Through controlling the squeezing pressure on the elastomeric female mold 14, it can be ensured that the elastomeric female mold deforms sufficiently to ensure good filling quality. The pressure sensor 19 may be disposed at the bottom of the support 111 and coupled to both the dispenser 16 and the support 111. However, the present invention is not limited to any particular position where the pressure sensor 19 is disposed. One skilled in the art can determine the predetermined pressure value by taking into account factors including the viscosity of the solution, the material of the elastomeric female mold and the shape and size of the cavities. For example, the predetermined pressure value can be determined as a value in the range of 20-40 N, preferably 20-30 N, more preferably, 30 N. The dispenser 16 may be threadedly coupled to the pressure sensor 19. The pressure sensor 19 may also be threadedly coupled to the support 111. As would be appreciated by those of ordinary skill in the art, the present invention is not limited to any particular coupling method between the dispenser 16 and the pressure sensor 19, and a suitable coupling may be established depending on the structure of the pressure sensor. Preferably, the apparatus 10 further includes a display device for displaying the pressure information.

The dispenser 16 further includes a liquid supply pipeline 168 in communication with the reservoir 161 for introducing the solution into the reservoir 161 from an external source. Preferably, the dispenser 16 further includes a liquid level sensor 169 (see FIGS. 4 and 5) disposed inside the reservoir 161 in order to sense a level of the solution in the reservoir 161 and produce liquid level information. Preferably, the liquid level sensor 169 is communicatively coupled to the controller 17, and the controller 17 is configured to receive the liquid level information and determined, based thereon, whether the current liquid level of the reservoir 161 is lower than a predetermined value. If so, the controller 17 issues alert information and/or cause the solution 18 to be bumped into the reservoir. More preferably, the level information may be displayed on the display device.

An exemplary process for manufacturing a microneedle by the above apparatus 10 equipped with the controller 17 includes the following steps.

At first, the controller 17 brings the dispenser 16 into the closed configuration. During use, the elastomeric female mold 14 is placed on the baseplate 15 and fixed by a fixture. As an example, the solution 18 is an aqueous low molecular weight (e.g., in the range of 300,000-1,000,000) sodium hyaluronate solution (e.g., with a dynamic viscosity of 10000 cps). The aqueous low molecular weight sodium hyaluronate solution is then bumped into the reservoir 161 through the liquid supply pipeline 168. The controller 17 controls the horizontal movement mechanism 12 and the vertical movement mechanism 11 to move so that the dispenser 16 reaches a suitable position where the roller 131 is in contact with the surface of the elastomeric female mold 14. Subsequently, under the control of the controller 17, the direct-acting solenoid 167 is energized, creating horizontal gaps between the two movable baffles 163 and the roller 131, and the height adjustment assembly is adjusted to create a predetermined gap between the surface of the elastomeric female mold 14 and the second baffle element 1632, which is equal to a desired coating thickness of the aqueous low molecular weight sodium hyaluronate solution to be coated on the surface of the elastomeric female mold 14. After the completion of the above adjustments, the controller 17 controls the vertical movement mechanism 11 to continue to move downward until a pressure sensed by the pressure sensor 19 reaches the predetermined pressure value. Following that, the vertical movement mechanism 11 is kept stationary. Afterwards, the controller 17 controls the horizontal movement mechanism 12 to make several linear reciprocal movements over a distance. The number and speed of the movements may depend on the properties of the solution and the size of the mold's cavities. Finally, the various components are returned to their respective initial positions, thus completing the filling of the aqueous low molecular weight sodium hyaluronate solution in the elastomeric female mold. After the aqueous low molecular weight sodium hyaluronate solution is dried, it is removed from the elastomeric female mold as the microneedle. In this manufacturing method, the resulting microneedle is an integrally-formed solid microneedle consisting of a substrate and microneedles. Apparently, the apparatus 10 can accomplish the manufacturing of the microneedle (in particular, a solid microneedle) without using the controller 17. In this case, the functions of the controller 17 are performed instead by manual intervention.

In other exemplary manufacturing processes, a smaller gap may be created between the surface of the elastomeric female mold 14 and the second baffle element 1632 by manipulating the height adjustment assembly. Moreover, after the various components have been returned to their respective initial positions and the filling of the aqueous low molecular weight sodium hyaluronate solution in the elastomeric female mold has been completed, the other aqueous low molecular weight sodium hyaluronate solution than that forms the microneedles may be removed (i.e., only the aqueous low molecular weight sodium hyaluronate solution that forms the microneedles is retained). Before or after it is dried, a substrate (formed otherwise optionally of a different material; in this case, the substrate and the microneedles may be formed separately; i.e., the formation of the substrate may either succeed or precede that of the microneedles) may be joined to the microneedles to produce the microneedle. In these cases, the resulting microneedle is a two-piece patch with the substrate and the microneedles as separate components.

The foregoing description presents merely some preferred embodiments of the present invention and is not intended to limit the scope of the present invention in any sense. It is intended that all changes and modifications made by those of ordinary skill in the art in light of the above teachings fall within the scope of the appended claims.

1. An apparatus for manufacturing a microneedle, the microneedle including a substrate and a number of needle bodies formed on the substrate, the apparatus for manufacturing a microneedle comprising:
a baseplate;
an elastomeric female mold disposed on the baseplate, the elastomeric female mold having a surface where cavities complementary to the needle bodies of the microneedle are formed;
a dispenser comprising a reservoir, the reservoir configured to contain a solution for manufacturing the microneedle;
a roller mechanism arranged on the dispenser, the roller mechanism comprising a roller rotatable about an axis of rotation;
a horizontal movement mechanism configured to drive the dispenser to move horizontally; and
a vertical movement mechanism configured to drive the dispenser to move vertically,
wherein: the reservoir comprises a closed configuration and an open configuration; when the reservoir is in the closed configuration, the reservoir encloses a space and prevents dispensing therefrom of the solution for manufacturing the microneedle, and when the reservoir is in the open configuration, the space of the reservoir is opened, allowing dispensing therefrom of the solution for manufacturing the microneedle;
when the reservoir is in the open configuration, the roller is configured to apply a predetermined value of pressure onto the elastomeric female mold under the drive of the dispenser that is being driven by the vertical movement mechanism and to then move horizontally on the elastomeric female mold and thus evacuate air from the cavities and coat the solution dispensed from the opened space on the elastomeric female mold under the drive of the dispenser that is instead being driven by the horizontal movement mechanism.

2. The apparatus for manufacturing a microneedle of claim 1, wherein the roller mechanism further comprises a roller drive assembly connected to the roller, the roller drive assembly configured to drive the roller to rotate about the axis of rotation, thus creating rolling friction between the roller and the elastomeric female mold.

3. The apparatus for manufacturing a microneedle of claim 2, further comprising a controller communicatively connected to each of the vertical movement mechanism, the horizontal movement mechanism, the roller mechanism and the dispenser, and wherein the controller is configured to control movement of each of the vertical movement mechanism, the horizontal movement mechanism, the roller mechanism and the dispenser.

4. The apparatus for manufacturing a microneedle of claim 1, wherein the dispenser is mounted on the vertical movement mechanism, the vertical movement mechanism is mounted on the horizontal movement mechanism, and
wherein the horizontal movement mechanism is configured to drive the vertical movement mechanism and the dispenser to move in a first horizontal direction which is perpendicular to both the axis of rotation of the roller and a vertical direction.

5. The apparatus for manufacturing a microneedle of claim 4, wherein the horizontal movement mechanism is further configured to drive the vertical movement mechanism and the dispenser to move in a second horizontal direction which is parallel to the axis of rotation of the roller.

6. The apparatus for manufacturing a microneedle of claim 1, wherein the horizontal movement mechanism comprises a horizontal movement member, a frame structure and a first driver, the frame structure disposed on the horizontal movement member, the horizontal movement member configured to be driven by the first driver to move horizontally together with the frame structure, the frame structure configured to cause horizontal movement of the dispenser.

7. The apparatus for manufacturing a microneedle of claim 6, further comprising a controller communicatively connected to the first driver, the controller configured to control a mode of operation of the first driver.

8. The apparatus for manufacturing a microneedle of claim 1, wherein the vertical movement mechanism comprises a vertical movement member, a second driver and a support, the support disposed on the vertical movement member, the vertical movement member configured to be driven by the second driver to move vertically together with the support, the support configured to cause vertical movement of the dispenser.

9. The apparatus for manufacturing a microneedle of claim 8, further comprising a controller communicatively connected to the second driver, the controller configured to control a mode of operation of the second driver.

10. (canceled)

11. The apparatus for manufacturing a microneedle of claim 1, wherein the horizontal and vertical movement mechanisms are gantry/carriage assemblies with X- and Z-directional movability, or parts of gantry/carriage assemblies with movability along three axes, and/or
wherein the dispenser is rotatably connected to the vertical movement mechanism.

12. The apparatus for manufacturing a microneedle of claim 1, wherein:
the dispenser comprises a top casing member, a fixed baffle and a plurality of movable baffles, the fixed baffle arranged adjacently to the movable baffle, the fixed baffle fixed to the top casing member, at least one of the movable baffles movably disposed on the top casing member, the roller rotatably arranged on the fixed baffle so as to be opposed to the top casing member, the top casing member, the fixed baffle, the roller and the movable baffles together forming the reservoir;
when the reservoir is in the closed configuration, each of the movable baffles is in sealed contact with the roller; and
when the reservoir is in the open configuration, at least one of the movable baffles movably disposed on the top casing member is spaced away from the roller.

13. The apparatus for manufacturing a microneedle of claim 12, comprising two movable baffles, which are disposed on opposite sides of the axis of rotation of the roller.

14. The apparatus for manufacturing a microneedle of claim 13, wherein the dispenser further comprises a horizontal adjustment assembly connected to at least one of the two movable baffles, the horizontal adjustment assembly configured to adjust a horizontal distance between the two movable baffles to cause a change in the configuration of the reservoir.

15. The apparatus for manufacturing a microneedle of claim 14, wherein the horizontal adjustment assembly comprises a biased element and a horizontal driver,
the horizontal driver configured to overcome a force exerted by the biased element to cause relative movement of the two movable baffles, the biased element configured to store potential energy during the relative movement of the two movable baffles caused by the horizontal driver, the biased element further configured to release the stored potential energy when the horizontal driver stops acting on the two movable baffles, thus cause reverse relative movement of the two movable baffles.

16. The apparatus for manufacturing a microneedle of claim 15, further comprising a controller communicatively connected to the horizontal driver, the controller configured to control a mode of operation of the horizontal driver, and/or
wherein the biased element is a compression spring and the horizontal driver comprises a direct-acting solenoid, the direct-acting solenoid comprising an output shaft connected to the movable baffle, the compression spring disposed over the output shaft of the direct-acting solenoid in such a manner that one end of the compression spring abuts against the direct-acting solenoid and the other end is connected to the movable baffle, or
wherein the dispenser further comprises a side casing member disposed outside the movable baffle, with the biased element being implemented as a compression spring and with the horizontal driver comprising a direct-acting solenoid, the direct-acting solenoid comprising an output shaft connected to one side of the movable baffle, the movable baffle connected at the opposite side to one end of the compression spring, the other end of the compression spring connected to the side casing member.

17-18. (canceled)

19. The apparatus for manufacturing a microneedle of claim 12, wherein the dispenser further comprises a height adjustment assembly connected to the movable baffles, the height adjustment assembly configured to adjust a vertical gap between at least one of the movable baffles and the elastomeric female mold, which defines a thickness of the substrate of the microneedle.

20. The apparatus for manufacturing a microneedle of claim 19, wherein the movable baffle comprises a first baffle element and a second baffle element, the second baffle element located closer to the elastomeric female mold, the second baffle element vertically movably disposed on the first baffle element, and wherein the height adjustment assembly is arranged on the first baffle element and configured to drive the second baffle element to move vertically, thus adjusting the vertical gap between the at least one movable baffle and the elastomeric female mold.

21. The apparatus for manufacturing a microneedle of claim 20, wherein the height adjustment assembly comprises a gear/rack drive mechanism for driving the second baffle element to move vertically relative to the first baffle element.

22. The apparatus for manufacturing a microneedle of claim 21, wherein the first baffle element has a cavity in which the gear/rack drive mechanism is accommodated, and an upper portion of the second baffle element is received in the cavity of the first baffle element and is connected to a rack in the gear/rack drive mechanism, or
wherein the first baffle element has a cavity and the gear/rack drive mechanism is arranged on an external side wall of the first baffle element spaced away from the reservoir, with an upper portion of the second baffle element being inserted in the first baffle element, or with the second baffle element being movably arranged adjacent to an internal side way of the first baffle element close to the reservoir, and/or
wherein the height adjustment assembly further comprises a height driver for electrically driving the gear/rack drive mechanism to move, or
wherein the height adjustment assembly further comprises a rotary adjustment knob disposed outside the first baffle element, the rotary adjustment knob connected to a gear in the gear/rack drive mechanism and configured to be manually manipulated to drive the gear/rack drive mechanism to move.

23-25. (canceled)

26. The apparatus for manufacturing a microneedle of claim 1, further comprising a pressure sensor for sensing pressure on the elastomeric female mold and producing pressure information for use in the determination of whether the pressure on the elastomeric female mold reaches a predetermined pressure value.

27. The apparatus for manufacturing a microneedle of claim 26, further comprising a controller communicatively connected to the pressure sensor,
the controller configured to determine, based on the pressure information, whether the pressure on the elastomeric female mold reaches the predetermined pressure value, and if so, control the vertical movement mechanism to stop moving downward, thereby keeping the current pressure on the elastomeric female mold.

28. A method for manufacturing a microneedle, which is based upon an apparatus for manufacturing the microneedle, the apparatus for manufacturing the microneedle comprising a baseplate, an elastomeric female mold, a dispenser, a roller mechanism, a horizontal movement mechanism and a vertical movement mechanism, the elastomeric female mold disposed on the baseplate, the elastomeric female mold having a surface where cavities complementary to needle bodies of the microneedle are formed, the dispenser comprising a reservoir for containing a solution for manufacturing the microneedle, the reservoir comprising a closed configuration and an open configuration, the roller mechanism arranged on the dispenser and comprising a roller rotatable about an axis of rotation, the method for manufacturing a microneedle comprising:
bringing the reservoir into the open configuration and thereby dispensing the solution for manufacturing the microneedle;
driving, by the vertical movement mechanism, the roller to vertically move downward and apply a predetermined value of pressure to the elastomeric female mold; and
driving, by the horizontal movement mechanism, the roller to horizontally move on the elastomeric female mold, thereby evacuating air from the cavities of the elastomeric female mold and coating the dispensed solution on the elastomeric female mold.

29. The method for manufacturing a microneedle of claim 28, further comprising:
monitoring the pressure on the elastomeric female mold and producing pressure information, by the pressure sensor, during the application of the pressure to the elastomeric female mold by the roller; and
controlling the pressure on the elastomeric female mold by causing the vertical movement mechanism, based on the pressure information, to continue or stop moving downward, and/or
wherein the dispenser comprises a top casing member, a fixed baffle and two movable baffles, at least one of the movable baffles movably disposed on the top casing member, the two movable baffles disposed on opposite sides of the roller, and
wherein the step of bringing the reservoir into the open configuration and thereby dispensing the solution for manufacturing the microneedle comprises:
driving the movable baffle movably disposed on the top casing member to move away from the roller, thus bringing the reservoir into the open configuration and allowing the solution to flow out of the reservoir onto the surface of the elastomeric female mold via a resulting gap between the movable baffle and the roller.

30-31. (canceled)

32. The method for manufacturing a microneedle of claim 28, further comprising, subsequent to the horizontal movement of the roller on the elastomeric female mold driven by the horizontal movement mechanism,
removing the other solution for manufacturing the microneedle than that that has formed the needle bodies and, before or after it is dried, joining a substrate to the needle bodies, thereby forming the microneedle, and/or
wherein the method further comprises, prior to the step of bringing the reservoir into the open configuration and thereby dispensing the solution for manufacturing the microneedle,
bringing the reservoir into the closed configuration and driving the dispenser by the horizontal movement mechanism and/or the vertical movement mechanism to move until the roller comes into contact with the surface of the elastomeric female mold.

33. (canceled)
